SEQUENCE LISTING (29 sequences)

SEQ ID NO: 1 - protein, 21 aa, Artificial Sequence
  Mouse IgK
METDTLLLWVLLLWVPGSTGD

SEQ ID NO: 2 - protein, 25 aa, Artificial Sequence
  human OSMMGVLLTQRTLLSLVLALLFPSMASM
MGVLLTQRTLLSLVLALLFPSMASM

SEQ ID NO: 3 - protein, 16 aa, Artificial Sequence
  VSV-G
MKCLLYLAFLFIGVNC

SEQ ID NO: 4 - protein, 19 aa, Artificial Sequence
  human IgG2 H
MGWSCIILFLVATATGVHS

SEQ ID NO: 5 - protein, 17 aa, Artificial Sequence
  BM40
MRAWIFFLLCLAGRALA

SEQ ID NO: 6 - protein, 21 aa, Artificial Sequence
  Secrecon
MWWRLWWLLLLLLLLWPMVWA

SEQ ID NO: 7 - protein, 22 aa, Artificial Sequence
  human IgKVIII
MDMRVPAQLLGLLLLWLRGARC

SEQ ID NO: 8 - protein, 16 aa, Artificial Sequence
  CD33
MPLLLLLPLLWAGALA

SEQ ID NO: 9 - protein, 23 aa, Artificial Sequence
  tPA
MDAMKRGLCCVLLLCGAVFVSPS

SEQ ID NO: 10 - protein, 18 aa, Artificial Sequence
  human chymotripsinogen
MAFLWLLSCWALLGTTFG

SEQ ID NO: 11 - protein, 15 aa, Artificial Sequence
  human trypsinogen 2
MNLLLILTFVAAAVA

SEQ ID NO: 12 - protein, 20 aa, Artificial Sequence
  human IL-2
MYRMQLLSCIALSLALVTNS

SEQ ID NO: 13 - protein, 17 aa, Artificial Sequence
  Gaussia luc
MGVKVLFALICIAVAEA

SEQ ID NO: 14 - protein, 16 aa, Artificial Sequence
  Albumin (HSA)
MKWVTFISLLFSSAYS

SEQ ID NO: 15 - protein, 16 aa, Artificial Sequence
  influence hemoagglutinin
MKTIIALSYIFCLVLG

SEQ ID NO: 16 - protein, 24 aa, Artificial Sequence
  human insulin
MALWMRLLPLLALLALWGPDPAAA

SEQ ID NO: 17 - protein, 11 aa, Artificial Sequence
  HIV-1 TATk
YARKAARQARA

SEQ ID NO: 18 - protein, 27 aa, Artificial Sequence
  MPG
GLAFLGFLGAAGSTMGAWSQPKKKRKV

SEQ ID NO: 19 - protein, 20 aa, Artificial Sequence
  PEP-1
KETWWETWWTEWSQPKKRKV

SEQ ID NO: 20 - protein, 22 aa, Artificial Sequence
  ARF(122)
MVRRFLVTLRIRRACGPPRVRV

SEQ ID NO: 21 - protein, 30 aa, Artificial Sequence
  BPrPr(130)
MVKSKIGSWILVLFVAMWSDVGLCKKRPKP

SEQ ID NO: 22 - protein, 18 aa, Artificial Sequence
  MAP
KLALKLALKALKAALKLA

SEQ ID NO: 23 - protein, 28 aa, Artificial Sequence
  Azurin p28
LSTAADMQGVVTDGMASGLDKDYLKPDD

SEQ ID NO: 24 - skipped

SEQ ID NO: 25 - protein, 6 aa, Artificial Sequence
  C105Y
PFVYLI

SEQ ID NO: 26 - protein, 6 aa, Artificial Sequence
  C105Y
PFVYLI

SEQ ID NO: 27 - protein, 22 aa, Artificial Sequence
  M918
MVTVLFRRLRIRRACGPPRVRV

SEQ ID NO: 28 - protein, 16 aa, Artificial Sequence
  DPV3
RKKRRRESRKKRRRES

SEQ ID NO: 29 - protein, 22 aa, Artificial Sequence
  human lactoferrin
KCFQWQRNMRKVRGPPVSCIKR

(19) United States
(12) Patent Application Publication
(10) Pub. No.: US 2022/0175966 A1
(43) Pub. Date: Jun. 9, 2022

(54) NEW GENE THERAPY CONSTRUCTS

(71) Applicants: ALMA MATER STUDIORUM - UNIVERSITÀ DI BOLOGNA, Bologna (IT); OREGON HEALTH & SCIENCE UNIVERSITY, Portland, OR (US)

(72) Inventors: Elisabetta CIANI, Bologna (IT); Hiroyuki NAKAI, West Linn, OR (US); Stefania TRAZZI, Bologna (IT); Claudia FUCHS, Bologna (IT)

(73) Assignees: ALMA MATER STUDIORUM - UNIVERSITÀ DI BOLOGNA, Bologna (IT); OREGON HEALTH & SCIENCE UNIVERSITY, Portland, OR (US)

(21) Appl. No.: 17/596,476

(22) PCT Filed: Jun. 1, 2020
(86) PCT No.: PCT/IB2020/055165

§ 371 Date: Dec. 10, 2021

(30) Foreign Application Priority Data
Jun. 13, 2019 (IT) 102019000008877

(51) Int. Cl. A61K 48/00; C12N 15/86

(52) U.S. Cl. CPC A61K 48/005; C12N 2710/14145; C12N 2710/14143; C12N 15/86

(57) ABSTRACT

The present invention provides a new vector for gene therapy said vector being therapeutically very efficient, viral particles comprising said vector, compositions comprising said viral particle, uses thereof, methods for the preparation of the vector), and therapies using said vector.

The present invention provides a new vector for gene therapy said vector being therapeutically very efficient, compositions comprising said vector, uses, methods for the preparation of the vector and therapies using said vector.

### DESCRIPTION

### State of the Art

Gene therapy can be broadly defined as the“transfer of genetic material” to cure, to prevent or to ameliorate a disease (the latter by at least to improving the clinical status of a patient). One of the basic concepts of gene therapy is to transform viruses into genetic shuttles, which will deliver the gene of interest into the target cells. Safe methods have been devised to do this, using several viral and non-viral vectors. Two main approaches emerged: in vivo modification and ex vivo modification. Retrovirus, adenovirus, adeno-associated virus are suitable for gene therapeutic approaches which are based on a permanent expression of the therapeutic gene. Gene therapy typically involves the insertion of a functioning gene into cells to correct a cellular dysfunction or to provide a new cellular function. Viruses that have been used to generate viral vectors are retrovirus, adenovirus, adenovirus associated virus (AAV), lentivirus, herpes simplex virus (HSV1), vaccinia virus.

Particularly suitable for gene therapy are monogenic diseases, i.e. disease caused by a single, nonworking or missing gene or gene pair. Gene replacement therapy is, in fact, being studied for monogenic diseases, as the function of only one gene needs to be fixed.

The use of viral vectors to deliver genes to patients affected with neurological disorders is an extremely attractive concept to researchers and clinicians.

As of December 2009, a total of 1579 gene therapy clinical trials have been initiated; a majority of these trials are Phase I, but only 3.6% of trials are Phase III.). Beyond, this is because treatment with gene therapy vectors have proven to have also several fatal drawbacks.

Until a decade ago, strategies for gene delivery to the brain were limited mostly to stereotaxic injection of viral vectors to the brain, and widespread gene delivery was achieved through the use of multiple injections to create pockets of transgene expression throughout the brain.

Recently, advancements in viral vector design and the exploration of alternative routes of administration have made global CNS gene delivery realistic. The most prominent CNS gene delivery vector is currently the adeno-associated virus (AAV). Several features make AAV vector an ideal vehicle with which to deliver genes to the CNS. Although AAV naturally infects humans, it is nonpathogenic and is classified as a dependovirus, because productive infection by AAV occurs only in the presence of a helper virus, either adenovirus or herpesvirus. In addition to its safety, AAV can infect both dividing and non-dividing cells and has the ability to confer long-term stable gene expression without causing associated inflammation or toxicity. Additionally, rapidly evolving vector production and purification methods facilitate both basic research applications as well as the possibility to produce the large quantities needed for clinical trials. Using AAV, advancements in global CNS gene delivery have accelerated to the point that treatments for neurodevelopmental disorders, such as lysosomal storage disease, Rett Syndrome, CDKL5 Deficiency Disorder (CDD), seem within reach. However, gene therapy is not without risks for the patient. In particular for CNS related disease, the major caveat regards the low efficiency of gene delivery to the CNS by viral vectors, thus requiring large vector doses and, consequently bringing the risk of immune reaction, as was the case in human clinical trials for hemophilia B (C. S. Manno et al., Successful transduction of liver in hemophilia by AAV-Factor IX and limitations imposed by the host immune response. Nat Med 12, 342-347 (2006); A. C. Nathwani et al., Long-term safety and efficacy of factor IX gene therapy in hemophilia B. N Engl J Med 371, 1994-2004 (2014)). Moreover, the new gene might be inserted in the DNA in the wrong location, possibly causing harmful mutations to the DNA, or even cancer, as shown in rodent studies (A. Donsante et al., AAV vector integration sites in mouse hepatocellular carcinoma. Science 317, 477 (2007)).

There is, therefore, a strong need to ameliorate the effectiveness of gene therapy while not increasing or even decreasing the strongly adverse side effects that are potentially triggered by the use of gene therapy vectors that are integrated in the host genome (which are the most effective in terms of therapeutic effect obtained).

### SUMMARY OF THE INVENTION

The authors of the present invention have provided modified gene therapy vectors comprising a first nucleic acid coding for a secretory leader sequence, operatively linked to a second nucleic acid coding for a protein transduction domain (PTD), operatively linked to a third nucleic acid coding for a therapeutic protein, that allows the reduction of viral load and/or number of infections needed in order to achieve the same effectiveness of the same vector carrying the therapeutic protein only.

The modified gene therapy vector of the invention provides an increased therapeutic activity compared to the same vector before the modification because the produced therapeutic protein, due to the secretory leader sequence is vehiculated outside the genetically modified cell, and, due to the PTD, is internalized in cells that are not genetically modified, thereby providing a therapeutic effect also in cells that have not been modified by the vector, hence amplifying, the therapeutic effect with respect to the number of cells infected.

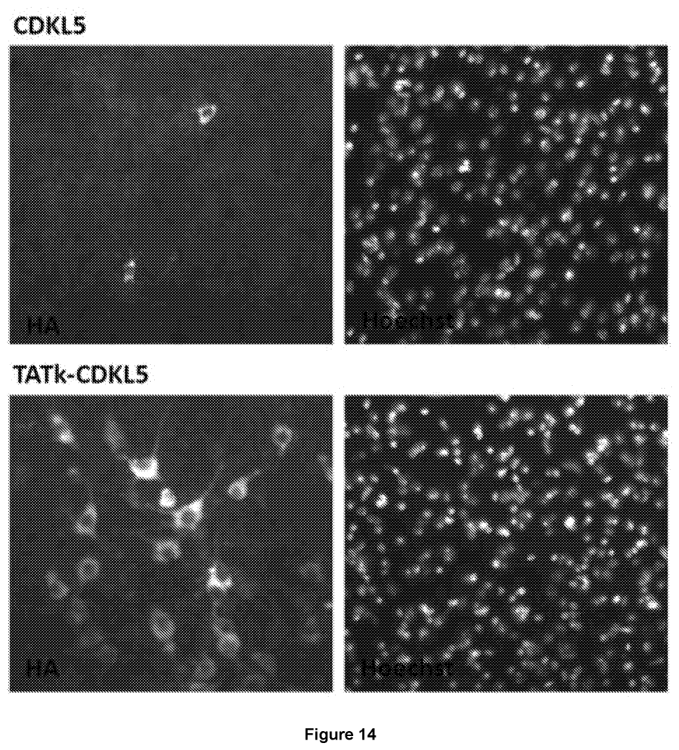

FIG. 14, described above, shows a dramatic increase in the amount of a therapeutic protein distribution into the CNS when the protein was produced by a vector modified according to the present invention in comparison with the non-modified vector.

Although the modified (AAV-leader-PTD-protein of interest) and the unmodified (AAV-protein of interest) showed a comparable infection efficiency, the number of cells positive for the therapeutic protein detectable in the CNS of the mice treated with the modified vector according to the claims was much higher (about 5-10 times) than that for therapeutic protein detected in mice treated with the unmodified vector.

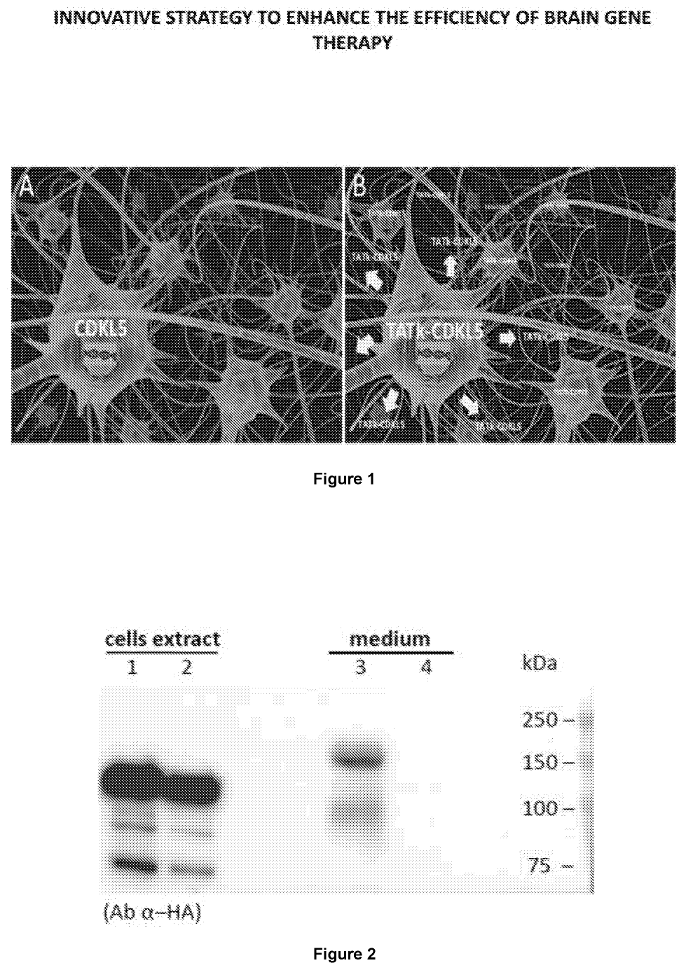

The technical consequence of this result is that less infections and/or a lower viral load are needed in order to obtain an ameliorated result in terms of amount and localization of the desired therapeutic protein, thereby drastically reducing the amount of transformed cells needed (which corresponds to a lower chance of side effects deriving from gene therapy such as cancer development and others) and providing a new tool, due to the enhanced effectiveness, for brain gene therapy. In practice, the protein produced by the infected cells is secreted and enters into neighbour cells, thereby amplifying the effect of the gene therapy because, even if the transduced cells are low in number, the transduced cells become a“factory” for the production of the therapeutic protein that is then distributed to neighbour cells (see FIG. 1). This dramatically lowers the number of cells that have to be transduced to provide the entire organ of interest, in particular CNS, with the secreted therapeutic factor and in this case, the efficiency of gene delivery does not necessarily need to be high. In turn, this allows the administration of small vector doses thus decreasing the risk of insertional mutagenesis and toxic side effects connected with large vector doses.

Therefore, object of the present invention is:

a gene therapy vector comprising a first nucleic acid coding for a secretory leader sequence, operatively linked to a second nucleic acid coding for a protein transduction domain (PTD), operatively linked to a third nucleic acid coding for a therapeutic protein; said gene therapy vector use in a gene therapy treatment; a gene therapy treatment wherein therapeutically effective doses of said vector are administered to a patient in need thereof;

### GLOSSARY

Gene therapy according to the present description has the meaning commonly recognized in the art, it therefore refers to a therapy through transfer of genetic material in the subject in need of a treatment, i.e. the therapeutic delivery of nucleic acid into a patient's cells as a drug to treat disease. Gene therapy according to the art and to the present invention can be achieved by transferring the genetic material of interest in the subject in need of treatment using a non-viral or a viral method. Viral vectors commonly used for human gene therapy include retroviruses, adenoviruses, herpes simplex virus, vaccinia virus, and adeno-associated virus. Viral vector genomes are either incorporated in the host's genome or stay as episomes.

Gene therapy vector according to the present invention refers to the nucleotide construct to be transferred in the patient's cell for gene therapy. The vector can be inserted in a viral particle that will consist of a viral capsid containing the nucleotide construct for gene therapy (i.e. the vector). In other terms, the vector according to the invention is the gene insert packaged inside a viral particle.

Adeno-associated virus (AAV) vector according to the present description has the meaning commonly recognized in the art, AAV vectors have been used in over 100 clinical trials for several diseases. AAV belongs to the parvoviruses. It is a single-stranded, non-enveloped DNA virus of 4.7 kb size, which causes a latent infection of human cells. Parvoviruses represent an alternative to malignancy-related retroviruses. Many naturally occurring AAV serotypes and variants have been isolated from various animal species including mammals, birds, and reptiles. Among them, the common AAV serotypes include AAV1, 2, 3, 4, 5, 6, 7, 8, 9, 10, 11, 12 and 13. Besides these serotypes, there are a number of AAV variants and mutants that have been used for AAV vector-mediated gene delivery, including AAV-DJ, AAV-LK03, AAV-PHP.B, AAV-PHP.eB, AAV-Retro, AAV2.7m8, among others. Typical AAV vectors comprise two ITR (inverted terminal repeat) regions, and within said ITRs at least the following elements: a promoter, a gene of interest and a terminator/polyadenylation signal.

Optimal serotype for brain gene therapy according to the present description refers to the AAV serotype which is best known in the art as to be more effective and suitable for brain gene therapy, i.e. to serotypes displaying strong neural tropisms. According to the present description, hence, the optimal serotype will be the serotype displaying the strongest tropism and infection efficiency depending on the target brain cell for therapy.

Viral particle according to the present description indicates a viral capsid containing a viral nucleotide construct (a viral vector), the viral particle according to the invention is also called in the art as“gene transfer vector”, and therefore consists of a viral particle containing a genetic construct that will be transferred in the infected cell.

According to the present description, the term monogenic disease has the meaning commonly used in the art and refers to result from modifications in a single gene occurring in all cells of the body. The mutation may be present on one or both chromosomes (one chromosome inherited from each parent). Examples of monogenic disorders are: sickle cell disease, cystic fibrosis, polycystic kidney disease, and Tay-Sachs disease.

Still, according to the description the term monogenic diseases also encompasses diseases in which the pathology can be caused by mutation of one or another gene in a group of genes. This means that when the pathology can be caused by mutations in different genes (one gene per patient), and therefore a single mutated gene in a group of identified genes, is responsible of the disease in a patient, the disease is considered a monogenic disease according to the present description.

Secretory leader sequence has the meaning commonly known in the art, and it refers to short peptides (leader sequences) present at the N-terminus of most newly synthesized proteins that are destined towards the secretory pathway. Several secretory leader sequences are known in the art.

Protein transduction domain or protein transmembrane domain (PTD) (or cell penetrating peptides CTPs) according to the present description has the meaning commonly known in the art, and it refers to small peptides able to carry proteins, peptides, nucleic acid, and nanoparticles, including viral particles, across the cellular membranes into cells. In general, PTDs can be classified into 3 types: cationic peptides of 6-12 amino acids in length, comprised predominantly of arginine, ornithine and/or lysine residues; hydrophobic peptides such as leader sequences of secreted growth factors and cytokines; and cell-type specific peptides, identified by screening of peptide phage display libraries.

### DETAILED DESCRIPTION OF THE FIGURES

FIG. 1

Comparison between the potential protein delivery of classical gene therapy (A) and innovative gene therapy (B) based on the insertion of a leader sequence for protein secretion from the infected cell plus a protein transduction domain in the target gene.

FIG. 2

Secretion of TATk-CDKL5 protein. Western blot analysis using an anti-HA antibody confirmed TATk-CDKL5, and CDKL5 protein expression in transfected HEK293T cells (cell extract, lanes 1-2) and TATk-CDKL5 protein accumulation in the concentrated (200×) culture medium (lane 3), indicating that the TATk-CDKL5 protein was secreted from HEK293T cells.

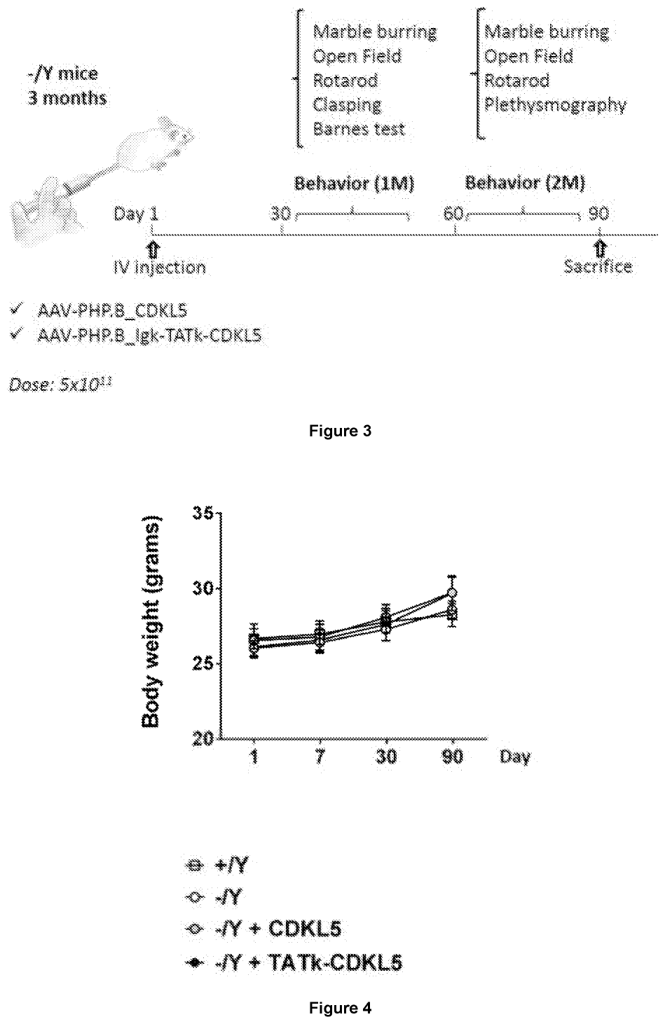

FIG. 3

Experimental plan. Adult mice (postnatal day 90, P90) were systemically treated (intravenous tail injection) with vehicle, AAVPHP.B_CDKL5 or AAVPHP.B_Igk-TATk-CDKL5 vectors, and brain samples were collected 90 days post injection.

FIG. 4

Body weight (in grams) of vehicle-treated wild-type (+/Y; n=13) and Cdkl5 KO (−/Y; n=15) and Cdkl5 KO mice treated with AAVPHP.B_CDKL5 or AAVPHP.B_Igk-TATk-CDKL5 vectors according to the treatment schedule shown in FIG. 3 (−/Y+CDKL5, n=10; −/Y+TATk-CDKL5; n=10). Mice were weighed before treatment (Day 1), and at 7, 30, and 90 days after treatment. Values represent mean±SEM.

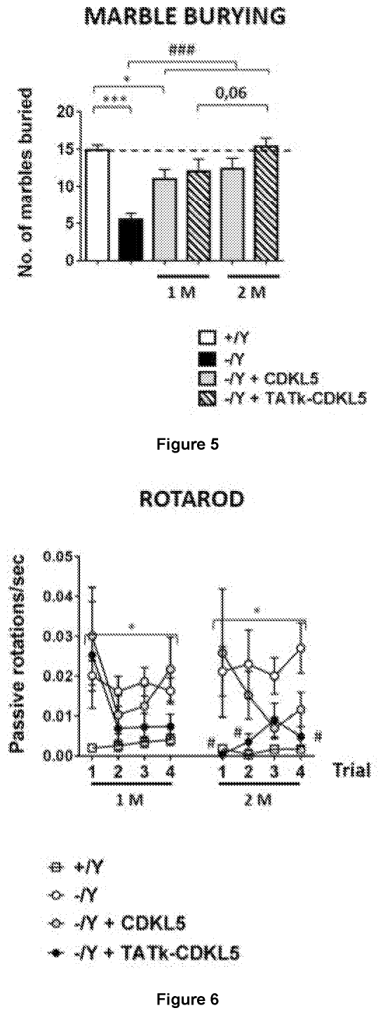

FIG. 5

Autistic-like features in treated Cdkl5 KO mice. Marble burying test: Cdkl5 KO mice (−/Y, n=17) buried fewer marbles compared to wild-type mice (+/Y, n=16). After 30 (1M) or 60 (2M) days from treatment with AAVPHP.B_CDKL5 (n=9) or AAVPHP.B_Igk-TATk-CDKL5 (n=9), Cdkl5 KO mice hid more marbles then vehicle-treated Cdkl5 KO mice. Values represent mean±SEM. *p<0.05 and ***p<0.001 as compared to the vehicle-treated wild-type condition; ###p<0.001 as compared to the vehicle-treated Cdkl5 KO samples (Fisher's LSD test after ANOVA).

FIG. 6

Impaired motor coordination in treated Cdkl5 KO mice. Rotarod assay, measuring frequency of passive rotations (rotations in which the mouse does not perform any coordinated movement but is passively transported from the rotating apparatus) on the accelerating rotating rod. Testing was performed in 4 trials with an inter-trial interval of 1 h. Mice were tested 30 and 60 days post treatment. Cdkl5 KO mice (−/Y, n=20) showed an increased frequency of passive rotations compared to wild-type mice (+/Y, n=20), indicating impaired motor coordination. Cdkl5 KO mice treated with AAVPHP.B_Igk-TATk-CDKL5 (n=9) showed an improvement in motor coordination at 60 days post treatment that was not present in Cdkl5 KO mice treated with AAVPHP.B_CDKL5 (n=9). Values represent mean±SEM. *p<0.05 as compared to the vehicle-treated wild-type condition; #p<0.05 as compared to the vehicle-treated Cdkl5 KO samples (Fisher's LSD test after ANOVA).

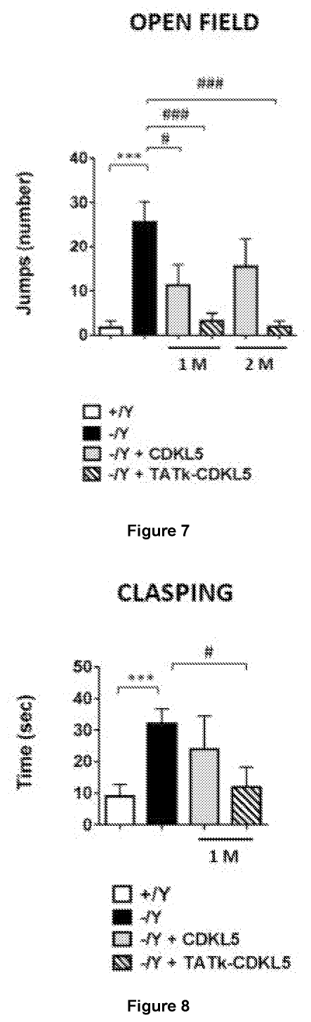

FIG. 7

Stereotypic behaviours in treated Cdkl5 KO mice measured as stereotypic jumps. Number of stereotypic jumps (repetitive beam breaks <1 s) in the corners of the open-field arena during the 20 min trial. Cdkl5 KO mice (−/Y, n=20) showed an increased number of repetitive stereotyped jumps compared to wild-type (+/Y, n=24) mice. Cdkl5 KO mice treated with AAVPHP.B_CDKL5 (n=9) or AAVPHP.B_Igk-TATk-CDKL5 (n=9) showed a drastic improvement of this behavior both after 30 (1M) and 60 (2M) days from treatment. Values represent mean±SEM. ***p<0.001 as compared to the vehicle-treated wild-type condition; #p<0.05, and ###p<0.001 as compared to the vehicle-treated Cdkl5 KO samples (Fisher's LSD test after ANOVA).

FIG. 8

Total amount of time spent hind-limb clasping during a 2 min interval in vehicle-treated wild-type (+/Y, n=14) and Cdkl5 KO (−/Y, n=24) mice, and in Cdkl5 KO mice treated with AAVPHP.B_CDKL5 (n=9) or AAVPHP.B_Igk-TATk-CDKL5 (n=9), 30 (1M) days from treatment. Values represent mean±SEM. ***p<0.001 as compared to the vehicle-treated wild-type condition; #p<0.05 as compared to the vehicle-treated Cdkl5 KO samples (Fisher's LSD test after ANOVA).

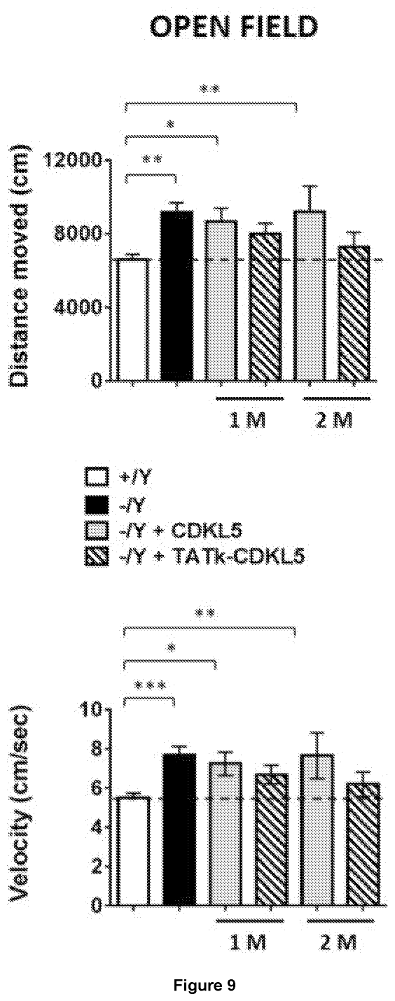

FIG. 9

Hyperactivity in treated Cdkl5 KO mice was measured as total distance travelled and average locomotion velocity during a 20 min open-field test. Cdkl5 KO mice (−/Y, n=24) exhibited increased locomotor activity with a longer total distance travelled at a greater average speed compared to wild-type (+/Y, n=24) mice. Cdkl5 KO mice treated with AAVPHP.B_CDKL5 (n=9) or AAVPHP.B_Igk-TATk-CDKL5 (n=9) showed an improvement of these behaviours both 30 (1M) and 60 (2M) days after treatment. Values represent mean±SEM. *p<0.05, **p<0.01 and ***p<0.001 as compared to the vehicle-treated wild-type condition (Fisher's LSD test after ANOVA).

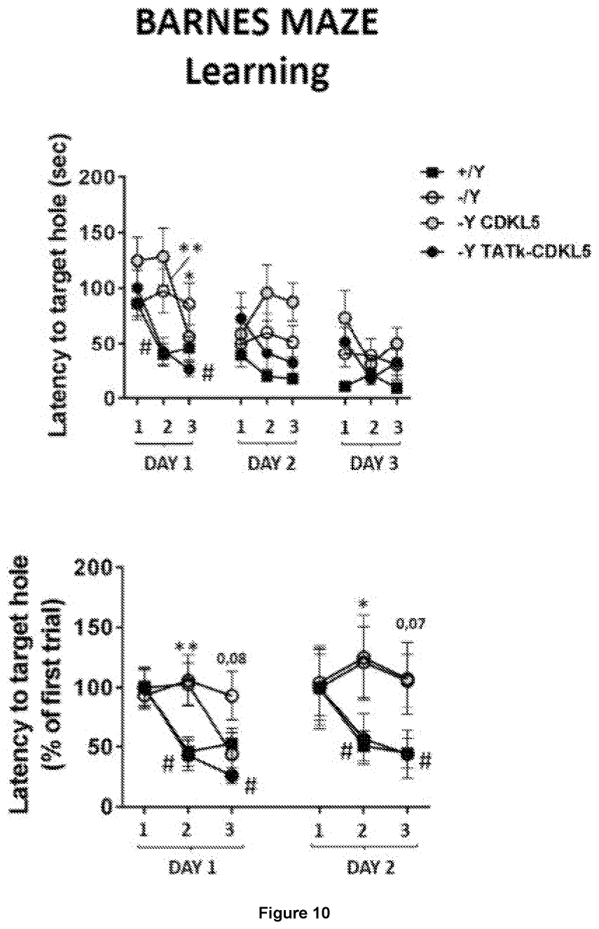

FIG. 10

Spatial learning and memory in treated Cdkl5 KO mice was assessed with the Barnes Maze. Graphs show the latency to find the target hole during the 3 trials a day for the 3-day learning period. Vehicle-treated Cdkl5 KO mice (−/Y, n=14) and Cdkl5 KO mice treated with AAVPHP.B_CDKL5 (n=9) showed an increased latency to find the target hole during the learning period compared to wild-type (+/Y, n=14) mice. Cdkl5 KO mice treated with AAVPHP.B_Igk-TATk-CDKL5 (n=8) showed an improvement in learning; this is particularly evident when the latency is expressed as a percentage of the first trial of each day (lower graph). Values represent mean±SEM. *p<0.05, **p<0.01 as compared to the vehicle-treated wild-type condition; #p<0.05 as compared to the vehicle-treated Cdkl5 KO samples (Fisher's LSD test after repeated ANOVA).

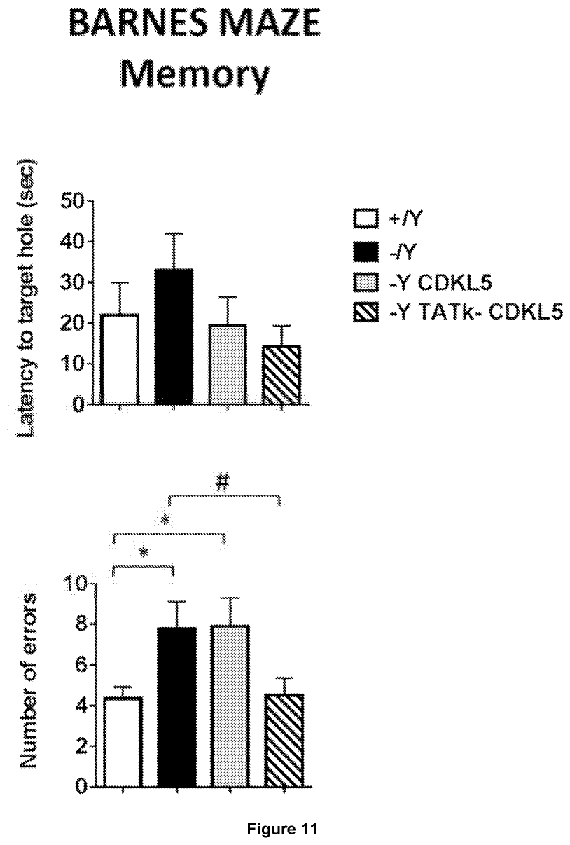

FIG. 11

Spatial memory assessed with the Barnes Maze in vehicle treated wild-type (+/Y; n=14) and Cdkl5 KO (−/Y; n=14) mice, and in Cdkl5 KO mice treated with AAVPHP.B_CDKL5 (n=9) or AAVPHP.B_Igk-TATk-CDKL5 (n=8). Graphs show the latency to find the target hole (upper histogram) and the number of errors before finding the target hole (lower histogram) on the probe day (day 4). Values represent mean±SEM. *p<0.05 as compared to the vehicle-treated wild-type condition. #p<0.05 as compared to the vehicle-treated Cdkl5 KO samples (Fisher's LSD test after repeated ANOVA).

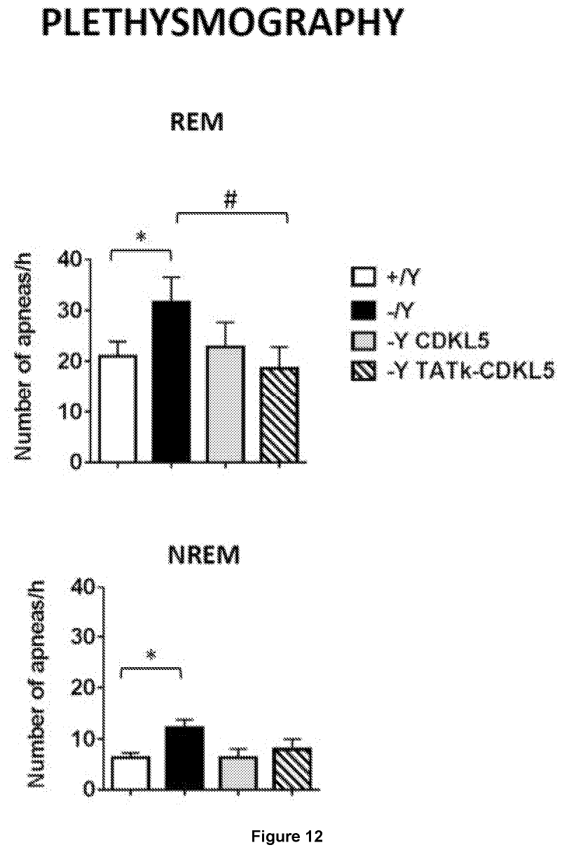

FIG. 12

Sleep apnoea occurrence rate in treated Cdkl5 KO mice was assessed using whole-body plethysmography. Sleep apnoea occurrence in vehicle-treated Cdkl5 KO (−/Y, n=12) and wild-type (+/Y, n=13) mice and in Cdkl5 KO mice treated with AAVPHP.B_CDKL5 (n=8) or AAVPHP.B_Igk-TATk-CDKL5 (n=8) during non-rapid eye movement (NREM) sleep, rapid eye movement (REM) sleep. *p<0.05 as compared to the vehicle-treated wild-type condition; #p<0.05 as compared to the vehicle-treated Cdkl5 KO samples (Fisher's LSD test after repeated ANOVA).

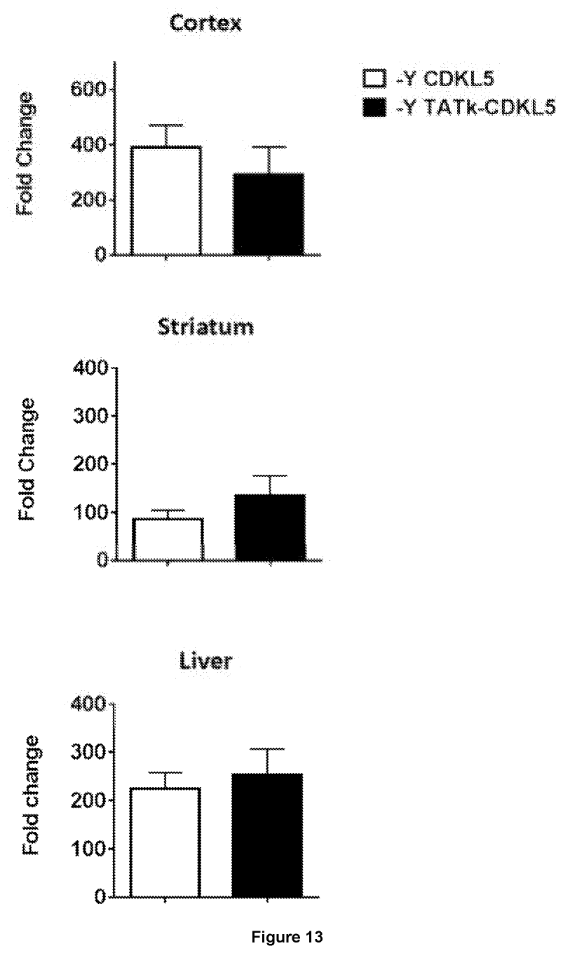

FIG. 13

Expression of mRNA for CDKL5 in cortex, striatum and liver from vehicle-treated Cdkl5 KO (−/Y, n=4) and from Cdkl5 KO mice treated with AAVPHP.B_CDKL5 (n=4) or AAVPHP.B_Igk-TATk-CDKL5 (n=4). Data are given as fold change of the Cdkl5 −/Y untreated condition.

FIG. 14

CDKL5 and TATk-CDKL5 protein distribution into the CNS. Images show TATk-CDKL5 or CDKL5 protein localization in the striatum of treated mice 90 days post injection. Localization of TATk-CDKL5 or CDKL5 was evaluated through immunohistochemistry using an anti-HA antibody, and nuclei were counterstained with Hoechst. Scale bar=70 □m.

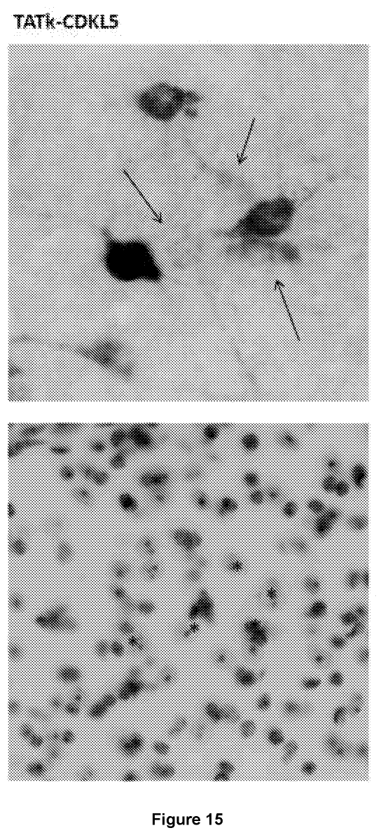

FIG. 15

TATk-CDKL5 protein distribution into the striatum. Images show TATk-CDKL5 protein localization in treated mice 90 days post injection. Localization of TATk-CDKL5 was evaluated through immunohistochemistry using an anti-HA antibody (upper panel), and nuclei were counterstained with Hoechst (lower panel). The arrows indicate the cells that probably received TATk-CDKL5 by local diffusion; the asterisks indicate the nuclei of the immunolabeled cells.

### DETAILED DESCRIPTION

The present invention hence provides a modified gene therapy vector, which increases the therapeutic effect of the same unmodified vector without substantially changing the infection efficiency of it.

The surprising technical effect observed, clearly visible in FIG. 14, is the dramatic increase in the amount of therapeutic protein distribution into the organ submitted to gene therapy with the modified vector according to the invention with respect to the amount of therapeutic protein distribution into the organ submitted to gene therapy with the non-modified vector. As stated above, the modified vectors of the invention provide an improved therapeutic effectiveness, and also allow, in order to obtain the same result obtainable with the same unmodified vector, a minor number of infection and/or a lower viral load per infection.

Due to reported negative side effects caused by the use of viral vectors for gene therapy (to note, the viral vector has the advantages of being more efficient and providing a time lasting transformation of the target cells with respect to the non-viral vectors), a viral vector for gene therapy that permits a decrease in the viral load and/or in the number of infections needed to obtain a therapeutic effect is extremely advantageous and dramatically increases the safety of the therapy.

In particular, a vector for gene therapy that substantially amplifies the therapeutic effect without need of amplifying the viral load and/or the number of infections (but rather allowing to decrease them) is particularly suitable for gene therapy in the CNS, in particular into the brain, due to the extremely low infection efficiency reported in the art for brain gene therapy.

An object of the invention is therefore a gene therapy vector comprising a first nucleic acid coding for a secretory leader sequence, operatively linked to a second nucleic acid coding for a protein transduction domain (PTD), operatively linked to a third nucleic acid coding for a therapeutic protein.

According to the description, any viral vector known for use in gene therapy is suitable to carry out the invention. Known viral gene therapy vectors include retroviruses, adenoviruses, herpes simplex, vaccinia, and adeno-associated virus.

A non-limiting example of suitable vectors according to the invention is represented by herpes simplex virus type 1 (HSV-1) vectors, lentiviral vectors, adenoviral vectors, and AAV vectors. AAV vectors are among the preferred vectors for carrying out the invention.

As the skilled person knows, AAV vectors are nonenveloped 25 nm particles with a foreign DNA packaging capacity of 4.7 kb. They have been clinically demonstrated to be safe in the various tissues. Several serotypes of AAV are known, each of them have displayed strong tropisms for certain organs or organ-specific cells.

According to the state of the art, the skilled person knows, e.g. the following:

| Tissue | Optimal Serotype |
|---|---|
| CNS | AAV1, AAV2, AAV4, |
| | AAV5, AAV8, AAV9 |
| Heart | AAV1, AAV8, AAV9 |
| Kidney | AAV2 |
| Liver | AAV7, AAV8, AAV9 |
| Lung | AAV4, AAV5, AAV6, AAV9 |
| Pancreas | AAV8 |
| Photoreceptor | AAV2, AAV5, AAV8 |
| Cells | |
| RPE (Retinal | AAV1, AAV2, AAV4, AAV5, AAV8 |
| Pigment | |
| Epithelium) | |
| Skeletal | AAV1, AAV6, AAV7, AAV8, AAV9 |
| Muscle | |

The skilled person is also well aware AAV-based vectors are derived from adeno-associated virus serotype 2, but also from other serotypes (AAV1, AAV3B, AAV4, AAV5, AAV6). Wild-type AAV2 is non-pathogenic and integrates site-specifically into the long arm of chromosome 19 after infection of human cells. Recombinant AAV2 vectors are generated by insertion of a therapeutic gene between two inverted terminal repeats (ITRs), replacing all coding sequences, including rep and cap genes, except ITRs. This recombinant AAV plasmid is co-transfected in HEK 293 cells with a“helper” plasmid containing rep and cap AAV genes and adenoviral E2A, E4, and VA genes needed for the appropriate expression of the AAV genes, but lacking the AAV inverted terminal repeats.

Therefore, the productive infection is dependent on the presence of a helper virus (adenovirus, herpes simplex virus, vaccinia virus). Rep and cap genes are necessary for replication and encapsidation, as well as for site-specific integration (Rep78 and Rep68 proteins). Consequently, because of the removal of all internal coding sequences, the recombinant AAV vectors lacking these genes are replication-defective unless co-transfected with a helper virus bearing these functions. The AAV-based vectors contain only a pair of 145 nucleotide-long ITRs of the original AAV genome and their packaging capacity for a DNA insert is 4-5 kb.

In one embodiment, the modified vector of the present invention is a gene therapy vector according to any of the embodiment described above wherein said vector is a vector that has an optimal serotype for CNS gene therapy.

As known in the art, when injected into the brain parenchyma, AAV serotypes display distinct properties that make each serotype more or less amenable to CNS gene delivery. AAV2 is the serotype used in most human clinical trials, mainly because it has been studied for the longest time and most detailed. AAV2 has a strong neuronal tropism but limited spread when injected intraparenchymally. AAV4 injected into the subventricular zone is reported to preferentially transduce astrocytes and ependymal cells. AAVS has been reported to transduce both astrocytes and neurons. The extent of AAVS's neuronal transduction is greater than that of AAV2, and AAVS will spread to a significantly larger volume. AAV1, AAV8, and AAV9 have primarily neuronal tropism, and when injected intraparenchymally they transduce significantly more cells than AAV2, with the following general efficiency for CNS delivery: AAV9>AAV8>AAV1. Therefore, according to the invention, vectors having an optimal serotype for CNS gene therapy are vectors that display a tropism for cells of the CNS. As stated above, any one of AAV1, AAV2, AAV4, AAVS, AAV8, and AAV9 is therefore suitable for carrying out this embodiment of the invention. The skilled person, depending on the target cells for the gene therapy, will have sufficient information in the art to select the optimal serotype for the desired cell therapy.

In an embodiment, the vector is selected among AAV1, AAV2, AAV4, AAVS, AAV8, AAV9, AAV10, AAV11, AAV12, and AAV13. In a further embodiment, the vector is selected from AAV9, AAV8 and AAV1, in a further embodiment, the vector is AAV9. As known by the skilled person, AAV vectors comprise two ITRs (inverted terminal repeats), and within said ITRs at least the following elements: a promoter, a gene of interest and a terminator/polyadenylation signal.

AAV gene therapy vectors according to the present invention therefore comprise or consist of a promoter, a secretory leader sequence, operatively linked to a second nucleic acid coding for a protein transduction domain (PTD), operatively linked to a third nucleic acid coding for a therapeutic protein, a polyadenylation signal between two inverted terminal repeats (ITRs).

Most AAV vectors in the art are designed with the AAV2 ITRs, however the invention is not limited to AAV2 ITRs although they are preferred.

The promoter according to the invention can be any suitable promoter, depending on the target of the therapy. Commonly used promoters include CMV (cytomegalovirus) promoter, EFla (elongation factor 1a) promoter, SV40 (simian virus 40) promoter, chicken β actin (CBA) and CAG (CMV, chicken β actin, rabbit β globin) promoters, CBh (CMV early enhancer fused to modified chicken β-actin) promoter that are constitutively active, however, also tissue specific promoters may be used in order to have a tissue specific expression. By way of example the neuron-specific enolase promoter can attain high levels of neuron-specific expression.

In a preferred embodiment, the CBh promoter, fully described in Gray et al Hum Gene Ther. 2011 September; 22 (9): 1143-115 is used.

In a preferred embodiment, the gene therapy vector of the invention comprises/consists of a nucleotide construct coding for an 800 nucleotides CBh promoter a first nucleotide sequence coding for a secretory leader sequence, operatively linked to a second nucleic acid coding for a protein transduction domain (PTD), operatively linked to a third nucleic acid coding for a therapeutic protein and a SV40 polyadenylation signal between two AAV2 inverted terminal repeats (ITRs).

CBh promoter is composed of: 1) GenBank accession no. NC_006273.2, nt 175625-175294 (CMV);.2) GenBank accession no. X00182.1, nt 280-540; 3) GenBank accession no. X00182.1, nt 544-676; and 4) GenBank accession no. NC_001510.1, nt 2312-2403 (VP intron). In an embodiment of the invention, the CBh promoter as disclosed in Gray et al. HUMAN GENE THERAPY 22:1143-1153 can be used.

According to the invention, the gene therapy vector comprises a first nucleotide sequence coding for a secretory leader sequence. The secretory leader sequence is a short peptide, and it can be any secretory leader sequence known in the art, capable of transporting the protein attached thereto outside a mammalian cell, in particular outside a human cell.

The skilled person knows several secretory leader sequences and can select the preferred one depending on the protein produced and on the target cell for gene therapy.

A non-limiting example of secretory leader sequences is provided in the table below.

TABLE 1

| Leader | | SEQ |
|---|---|---|
| sequence | | ID |
| Name | Sequence | NUMBER |
| Mouse IgK | METDTLLLWVL | 1 |
| | LLWVPGSTGD | |
| Human OSM | MGVLLTQRTLL | 2 |
| | SLVLALLFPSM | |
| | ASM | |
| VSV-G | MKCLLYLAFLF | 3 |
| | IGVNC | |
| Human IgG2 H | MGWSCIILFLV | 4 |
| | ATATGVHS | |
| BM40 | MRAWIFFLLCL | 5 |
| | AGRALA | |
| Secrecon | MVWVRLWWLLL | 6 |
| | LLLLLWPMVWA | |
| Human IgKVIII | MDMRVPAQLLG | 7 |
| | LLLLWLRGARC | |
| CD33 | MPLLLLLPLLW | 8 |
| | AGALA | |
| tPA | MDAMKRGLCCV | 9 |
| | LLLCGAVFVSP | |
| | S | |
| Human | MAFLWLLSCWA | 10 |
| Chymotrypsinogen | LLGTTFG | |
| Human | MNLLLILTFVA | 11 |
| trypsinogen-2 | AAVA | |
| Human IL-2 | MYRMQLLSCIA | 12 |
| | LSLALVTNS | |
| Gaussia luc | MGVKVLFALIC | 13 |
| | IAVAEA | |
| Albumin(HSA) | MKWVTFISLLF | 14 |
| | SSAYS | |
| Influenza | MKTIIALSYIF | 15 |
| Haemagglutinin | CLVLG | |
| Human insulin | MALWMRLLPLL | 16 |
| | ALLALWGPDPA | |
| | AA | |

Any nucleotide sequence coding for the amino acid sequences above can be inserted in the vector of the invention.

The sequence of each of said exemplified secretory leader peptides is known in the art and therefore a nucleic acid coding for each of said sequences can be easily designed.

In a preferred embodiment, the secretory leader sequence is mouse Igk.

Further, the vector of the invention comprises, operatively linked to the nucleic acid coding for the secretory leader sequence, a protein transduction domain or PTD. Several PTDs are known in the art, and the skilled person, also in this case, would have no difficulties in selecting the desired one, also depending on the therapeutic protein expressed and on the target cells. In any case, a non-limiting example of PTDs suitable for carrying out the invention is TATk, MPG, Pep-1, ARF(1-22), BPrPr(1-30), MAP, p28, VT5, C105Y, M918, DPV3, Human lactoferrin, an example of suitable PTD amino acid sequences is depicted in Table 2 below:

| | | SEQ ID |
|---|---|---|
| PTD Name | Sequence | NUMBER |
| HIV-1 TATk | YARKAARQARA | 17 |
| MPG | GLAFLGFLGAA | 18 |
| | GSTMGAWSQPK | |
| | KKRKV | |
| PEP-1 | KETWWETVWVT | 19 |
| | EWSQPKKRKV | |
| ARF(1-22) | MVRRFLVTLRI | 20 |
| | RRACGPPRVRV | |
| BPrPr(1-30) | MVKSKIGSWIL | 21 |
| | VLFVAMWSDVG | |
| | LCKKRPKP | |
| MAP | KLALKLALKAL | 22 |
| | KAALKLA | |
| Azurin p28 | LSTAADMQGVV | 23 |
| | TDGMASGLDKD | |
| | YLKPDD | |
| VT5 | DPKGDPKGVTV | 25 |
| | TVTVTVTGKGD | |
| | PKPD | |
| C105Y | PFVYLI | 26 |
| M918 | MVTVLFRRLRI | 27 |
| | RRACGPPRVRV | |
| DPV3 | RKKRRRESRKK | 28 |
| | RRRES | |
| Human | KCFQWQRNMRK | 29 |
| lactoferrin | VRGPPVSCIKR | |

Any nucleotide sequence coding for the amino acid sequences above can be inserted in the vector of the invention.

The sequence coding the indicated PTD can, if necessary, be modified in the part coding for the furin recognition sequence to avoid cleavage and loss of the PTD domain during its secretory transition through the endoplasmic reticulum and Golgi as explained in“Delivery of therapeutic proteins as secretable TAT fusion products” Mol Ther. 2009 February; 17 (2):334-42. doi: 10.1038/mt.2008.256. Epub 2008 Dec. 2.

According to the invention any of the vectors listed above can comprise a nucleic acid coding for one of any of the secretory leader sequences listed above, operatively linked to a nucleic acid coding for any of the PTDs listed above.

In a preferred embodiment, an AAV vector comprising a sequence coding mouse Igk operatively linked to a sequence coding TATk will be used.

The gene therapy vectors of the invention can code for any desired therapeutic protein, in particular for proteins related to monogenic disorders. In a preferred embodiment, the protein is a protein suitable for CNS gene therapy.

A non-limiting example of said proteins is CDKL5, MECP2, FOXG1, SCN1A, NLGN3, SHANK3, ASPA, PAH.

The skilled person can easily find the necessary information for the desired sequence in genbank, by way of example, the proteins indicated above are reported with their genbank accession number in Table 3 below.

| Gene Name | Gene ID | MIM |
|---|---|---|
| Cyclin dependent kinase like 5 (CDKL5) | 6792 | 300203 |
| Methyl-CpG binding protein 2 (MECP2) | 4204 | 300005 |
| Forkhead box G1 (FOXG1) | 2290 | 164874 |
| Sodium voltage-gated channel | 6323 | 182389 |
| alpha subunit 1 (SCN1A) | | |
| Neuroligin 3 (NLGN3) | 54413 | 300336 |
| SH3 and multiple ankyrin repeat | 85358 | 606230 |
| domains 3 (SHNK3) | | |
| Aspartoacylase (ASPA) | 443 | 608034 |
| Phenylalanine hydroxylase (PAH) | 5053 | 612349 |

The vector in any embodiment or in any combination of embodiment described above can be used for gene therapy, in particular for CNS gene therapy, in particular for brain gene therapy.

The gene therapy wherein the optimized vector of the invention is delivered to the target cells of the patient can be carried out both for therapies of multigenic and monogenic diseases.

In case more the disease is caused by the coexistence of mutations of different genes (more than one) (of course in a number that the skilled person would deem still suitable for gene therapy through viral infection), either a mixture of modified vectors according to the invention coding for different therapeutic proteins could be used, or multiple infection events for each therapeutic modified vector could be carried out.

The skilled person would know which protocol is more suitable depending on the disease and on the general health conditions of the patient.

In case a disease is defined as polygenic or multigenic merely due to the fact that the same pathology can be caused by mutations in different genes, and therefore the same pathological phenotype is caused by a different pathological genotype in which mutation of either one or another gene in a defined group of genes can cause the same pathology, due to the fact that the cause of the pathology in a single patient is caused by mutation of a single gene (in homozygosis or in heterozygosis), in the meaning of the present description the pathology still falls in the definition of “monogenic” disease. In fact, in this case, the same symptoms (that fall into the definition of the same disease according to medicine) can be considered, genetically, as a sum of different genetic disorders all providing the same pathological phenotype and therefore defined by medical doctors with the same pathology name. A long as a single therapeutic protein is suitable for treating the disease in a single patient, the disease falls, according to the present description, in the definition of monogenic disease.

At present gene therapy is preferentially used to treat (or to try to treat) monogenic diseases. Therefore, according to a preferred embodiment, the uses indicated above are applied to monogenic diseases and herein defined.

A non-limiting example of diseases that can be treated by gene therapy with the gene therapy vector of the invention is Dravet syndrome, Phenylketonuria, ASD, Phelan-McDermid syndrome, Canavan disease or Rett Syndrome. Therefore, according to the description, depending on the protein encoded by the third nucleic acid in the vector of the invention, any of the above-mentioned diseases can be treated.

All diseases, wherein the administration of a replacement protein has proven useful are suitable for gene therapy, as well as all diseases in which a protein mutation or deletion is known to be a direct cause of the disease, can be eligible for gene therapy.

The skilled person can readily find all proteins whose mutations or deletions are related to genetic diseases and therefore, depending on the protein selected, the vector of the invention can be used to treat a desired disease.

Multiple routes of vector administration have been explored to target specific disease application:
Direct injection offers a simple but invasive means to efficiently transduce a small area
Injection into the CSF within the brain allows broad distribution of the vector across the CNS, with limited penetration into the brain parenchyma
Further intrathecal injection via a lumber puncture distributes the vector through the CSF, but mostly targets the spinal cord and dorsal root ganglia (DRGs).
Intramuscular injection targets motor and sensory neurons via retrograde transport
Intravascular administration can achieve widespread transduction throughout the CNS, but requires high vector doses and peripheral exposure to the vector. Any of the administration routes indicated above can be used to carry out the gene therapy with the vector of the invention.

The vector of the invention can be prepared according to the techniques commonly used in the art. In general, the most widely used platform for producing AAVs (such as modified/recombinant AAVs) involves transfecting HEK293 cells with either two or three plasmids, one encoding the gene of interest, one coding the AAV rec/cap genes and another containing helper genes provided by ether adeno or herpes viruses. The production can be carried out with adherent cells or with suspension-adapted HEK293 cells. In other AAV manufacturing platforms one or more genetic components for AAV manufacturing are integrated into the genome of mammalian or insect production cells.

Protocols for producing AAV vectors are known in the art, any of the known protocols can be used to carry out the invention.

In order to carry out gene therapy, the vector of the invention has to be delivered to the target cells. The vector will therefore be packaged in a viral particle (i.e. a suitable capsid containing the vector) suitable for gene therapy. The capsid will be selected according to the viral vector used. Therefore, another object of the invention is represented by a viral particle consisting of a viral capsid and a gene therapy vector comprising a first nucleic acid coding for a secretory leader sequence, operatively linked to a second nucleic acid coding for a protein transduction domain (PTD), operatively linked to a third nucleic acid coding for a therapeutic protein, according to any of the embodiments described above.

In a preferred embodiment, the viral particle will be an AAV particle, therefore a particle consisting of capsid is an AAV capsid and an adeno-associated viral vector (AAV) in any of the embodiments defined above.

Therefore, in one embodiment of the invention said vector has an optimal serotype for CNS gene therapy as previously described.

For AAV vectors, several AAV capsid proteins are known in the art, and several AAV capsids can be designed depending on the target cells for the therapy.

AAV is known to enter cells through interactions with carbohydrates present on the surface of the target cells, typically sialic acid, galactose and heparin sulfate. It is known that differences in sugar-binding preferences encoded in capsid sequence can influence cell-type transduction preferences of the various AAV.

AAV9 preferential galactose binding is believed to confer to the virus the unique ability to cross the blood-brain barrier (BBB) and infect the cells of the CNS including primary neurons.

According to the invention, a suitable AAV capsid is one of AAV-PHP, AAV9, AAV-BR1, AAV-Retro capsid.

In an embodiment of the invention said AAV-PHP capsid is AAV-PHP.B, AAV-PHP.eB, AAV-PHP.S or AAV-PHP.A. AAV-PHP capsid proteins (and related capsids) are well-known genetically engineered AAV capsid protein created based on AAV serotype 9 capsid.

According to the invention, the vector packaged into the viral particle codes for a therapeutic protein, in a particular embodiment, said therapeutic protein is a protein for CNS therapy as already defined above.

A non-limiting example of said therapeutic protein comprises CDKL5, MECP2, FOXG1, SCN1A, NLGN3, SHANK3, ASPA, PAH.

The viral particle in any of the embodiments or examples herein provided, is intended in particular for a gene therapy treatment, in other words, is intended for use in a gene therapy treatment as already described in the present specification with reference to the gene therapy vector above.

All definitions and examples of gene therapy methods/uses provided for the gene therapy vector of the invention apply, mutatis mutandis, to the viral particle of the invention.

Another object of the invention is a pharmaceutical composition comprising or consisting of the viral particle in any of the embodiments described above and a suitable pharmaceutically acceptable carrier. The composition is a composition suitable for systemic (including intravenous) injection, central nervous system delivery or aerosol/nasal delivery. By way of example the injection can be intravenous injection, intraparenchymal administration in particular areas of the brain such as intracerebroventricular, cisternal, lumbar or intrathecal administration, or intra-arterial injection (carotid artery injection may be used for the delivery), or by direct administration into the cerebrospinal fluid.—

Suitable pharmaceutical carriers for the administrations indicated above are well-known to the skilled person, by way of example saline solution can be used.

When intended for medical treatment every product herein described will be in sterilized form.

A further object of the invention is therefore a medical treatment comprising administering therapeutically effective doses of the viral particle or of the pharmaceutical composition of the invention to a patient in need thereof.

All the embodiment provided in the description related to the vector and the disease to be treated by using the vector apply to the viral particle, to the pharmaceutical composition as well as to the use or to the medical treatment according to the invention. Comprising the administration routes.

The following examples are focused on one specific embodiment of the invention, that is to say an AAV modified vector according to the invention, wherein the operatively linked three nucleotide sequences coding for the secretory leader sequence-PTD-gene of interest code, respectively, for IgK-TATκ-CDKL5. These examples are provided to show completely one way to carry the invention and the observed results in vivo, however, it is obvious that they do not intend to limit in any aspect the claims or the embodiments of the invention as different leader sequences, PTDs and genes can be used, following the teachings in the descriptions and in the examples, in order to carry out the invention claimed.

All experiments on animals were performed in accordance with the Italian and European Community law for the use of experimental animals and were approved by Bologna University Bioethical Committee.

### EXAMPLES

### General Protocol for AAV Viral Particle Production

AAV vectors were produced in HEK 293 cells by an adenovirus-free plasmid transfection method (Matsushita T, Elliger S, Elliger C, Podsakoff G, Villarreal L, Kurtzman G J, Iwaki Y, Colosi P. 1998. Adeno-associated virus vectors can be efficiently produced without helper virus. Gene Ther 5:938-945) with modification on a scale of 1 to 2-liter culture. If brief, HEK 293 cells (AAV-293) were purchased from Agilent (AAV-293 cells) and were grown in Dulbecco's modified Eagle's medium (DMEM) (Lonza, Basel, Switzerland) supplemented with 10% fetal bovine serum (FBS), L-glutamine, and penicillin-streptomycin. Immediately before plasmid DNA transfection, the culture media were changed to serum-free media. The following three plasmids were transfected at a 1:1:1 ratio in HEK293 cells using the standard polyethyleneimine (PEI) (1 mg/mL) DNA transfection procedure at a DNA:PEI weight ratio of 1:2. The three plasmids used for AAV vector production were: (1) pHELPER (an adenovirus helper plasmid from Agilent), (2) pHLP-AAV-PHP.B (an AAV helper plasmid supplying AAV2 Rep and AAV-PHP.B capsid proteins, constructed based on an AAV9 helper plasmid obtained from James M. Wilson, University of Pennsylvania), and (3) an AAV vector plasmid containing a transgene expression cassette placed between the two AAV2 inverted terminal repeats (ITRs), constructed based on an AAV vector plasmid obtained from Avigen Inc. Five days after transfection, both media and cells were harvested. The harvested media and cells underwent one cycle of freezing and thawing and the cell debris was removed by centrifugation. The culture medium supernatants were made to 8% polyethylene glycol (PEG) 8000 and 0.5 M NaCl, incubated on ice for 3 h, and spun at 10,000×g for 30 min to precipitate viral particles. The pellets were resuspended in a buffer containing 50 mM Tris-HCl (pH 8.5) and 2 mM MgCl2, treated with Benzonase (EMD Millipore, Darmstadt, Germany) at a concentration of 200 units per mL for 1 h, and subjected to purification by two rounds of cesium chloride (CsCl) density-gradient ultracentrifugation (Grimm D, Zhou S, Nakai H, Thomas C E, Storm T A, Fuess S, Matsushita T, Allen J, Surosky R, Lochrie M, Meuse L, McClelland A, Colosi P, Kay M A. 2003. Preclinical in vivo evaluation of pseudotyped adeno-associated virus vectors for liver gene therapy. Blood 102:2412-2419) followed by dialysis with phosphate-buffered saline (PBS) with 0.001% Pluronic F68. The final viral preparations were made in PBS/5% sorbitol/0.001% Pluronic F68 and stored −80oC until use. The AAV vector titers were determined by a quantitative dot blot assay Powers J M, Chang X L, Song Z, Nakai H. 2018. A Quantitative Dot Blot Assay for AAV Titration and Its Use for Functional Assessment of the Adeno-associated Virus Assembly-activating Proteins. J Vis Exp12 doi:10.3791/56766).

### Production of AAV Vector for Gene Therapy

To express the IgK-TATκ-CDKL5 and CDKL5 proteins in neurons by means of AAV vector-mediated gene delivery, an IgK-TATκ-CDKL5 and CDKL5 gene expression cassette under the control of a strong, long-term, and ubiquitous CNS expression promoter (CBh, S. J. Gray et al., Optimizing promoters for recombinant adeno-associated virus-mediated gene expression in the peripheral and central nervous system using self-complementary vectors. Hum Gene Ther 22, 1143-1153 (2011)) inserted in the AAV vector plasmid with which we can produce recombinant AAV vectors was constructed. The expression cassettes contain a 800-bp CBh promoter, the IgK-TATκ-CDKL5 (3.2 kb) or CDKL5 (3.1 kb) open reading frame and a 0.1-kb SV40 polyadenylation signal between the two AAV2 inverted terminal repeats (ITRs).

### Recombinant AAV Production

Recombinant AAV vectors used the AAV-PHP.B capsid (B. E. Deverman et al., Cre-dependent selection yields AAV variants for widespread gene transfer to the adult brain. Nat Biotechnol 34, 204-209 (2016)) and were produced by a triple-transfection method in human embryonic kidney 293 (HEK293) cells as described (Grieger et al. Separate basic region motifs within the adeno-associated virus capsid proteins are essential for infectivity and assembly. J Virol. 80, 5199-210 (2006).). In brief, we changed the complete culture medium to serum-free medium immediately before transfection, transfected cells with a mixture of the required amount of each plasmid DNA with polyethyleneimine (PEI) at a DNA/PEI weight ratio of 1:2, and harvested both medium and cells for viral particle recovery at 5 days post transfection. The AAVPHP.B_CDKL5 and AAVPHP.B_Igk-TATk-CDKL5 vectors were produced by using 25 to 50 225-cm2 flasks. The harvested medium (1 L to 2 L) and cells underwent one cycle of freezing and thawing, and the cell debris was removed by centrifugation at 10,000×g for 15 min. The culture medium supernatants were made with 8% polyethylene glycol 8000 (PEG 8000) and 0.5 M NaCl, incubated on ice for 3 h, and spun at 10,000×g for 30 min to precipitate viral particles. The pellets were resuspended in a buffer containing 50 mM Tris-HCl (pH 8.5) and 2 mM MgCl2, treated with Benzonase (EMD Millipore, Darmstadt, Germany) at a concentration of 200 U per ml for 1 h, and subjected to purification by two rounds of CsCl density gradient ultracentrifugation (J. M. Powers, X. L. Chang, Z. Song, H. Nakai, A Quantitative Dot Blot Assay for AAV Titration and Its Use for Functional Assessment of the Adeno-associated Virus Assembly-activating Proteins. J Vis Exp, (2018)) followed by dialysis with a buffer (phosphate-buffered saline (PBS) containing 5% d-sorbitol).

### Animal Husbandry and Treatments

The mice used in this work derive from the Cdkl5 null strain in C57BL/6N background developed in E. Amendola et al., Mapping pathological phenotypes in a mouse model of CDKL5 disorder. PLoS one 9, e91613 (2014) and backcrossed in C57BL/6J for three generations. Cdkl5+/Y mice and Cdkl5 −/Y littermate control mice were used for all experiments. The day of birth was designated as postnatal day (P) zero and animals with 24 h of age were considered as one-day-old animals (P1). After weaning, mice were housed 3-5 per cage on a 12 h light/dark cycle in a temperature—(23° C.) and humidity-controlled environment with food and water provided ad libitum. The animals' health and comfort were controlled by the veterinary service. Experiments were performed in accordance with the Italian and European Community law for the use of experimental animals and were approved by Bologna University Bioethical Committee. In this study, all efforts were made to minimize animal suffering and to keep the number of animals used to a minimum.

To test the efficacy of CDKL5 gene therapy we treated adult (P60) Cdkl5 KO (−/Y) mice. Adult mice received 5×1011 viral genome (vg) particles of AAVPHP.B in 0.2 mL via the lateral tail vein (intravenous injection, IV) (FIG. 3). The effects of treatment were evaluated in Cdkl5 KO mice 30 and 60 days post-injection, see Treatment schedule (FIG. 3). Ninety days after the viral injection, mice were sacrificed and CDKL5 or GFP protein distribution was evaluated by immunohistochemistry. This experimental design allowed us to study the effect of gene therapy on the same animal at different times after the viral injection.

### Vector Biodistribution

Tissue DNA was extracted with QlAamp DNA Mini Kit (QIAGEN, CA), and vector genomes were quantified by real-time PCR using SsoAdvanced Universal SYBR Green Supermix (Bio-Rad, CA, USA) and primer/probe targeting the CBh2 promoter sequence of the vectors and the mouse agouti gene. Mouse-agouti Fw 5′-GGCGTGGTCAGTGGTTGTG-3′, Rv 5′-TTTAGCTTCCACTAGGTTTCCTAGAAA-3′; CBh2 Fw 5′-TACTCCCACAGGTGAGCGG-3′, Rv 5′-GGCAGGTGCTCCAGGTAAT-3′. Copy number is reported as vector genomes per microgram of genomic DNA.

### Quantitative Real Time PCR and Standard Reverse Transcription-PCR

Total RNA was isolated from the striatum, cortex and liver of Cdkl5 KO mice treated with vehicle, AAVPHP.B_CDKL5 (n=4) or AAVPHP.B_Igk-TATk-CDKL5 (n=4) with TRI reagent (Sigma-Aldrich, MO, USA) according to the manufacturer's instructions. cDNA synthesis was achieved with 5 μg of total RNA using iScript™ Advanced cDNA Synthesis Kit (Bio-Rad, CA, USA), according to the manufacturer's instructions. We used the primers that gave an efficiency that was close to 100%. Real-time PCR was performed using SsoAdvanced Universal SYBR Green Supermix (Bio-Rad, CA, USA) in an iQ5 Real-Time PCR Detection System (Bio-Rad, CA, USA). The primer sequences used were as follows: glyceraldehyde-3-phosphate dehydrogenase (GAPDH; NM_008084.2), Fw 5′-GAACATCATCCCTGCATCCA-3′, Rv 5′-CCAGTGAGCTTCCCGTTCA-3′; Fw (span exons 3-4): 5′-GCAGACACAAGGAAACACATGA-3′, Rv 5′-CAACTTTCCCCTCCGACGAA-3′ Relative quantification was performed using the ΔΔ Ct method.

### Histology

Mice were deeply anesthetized with isoflurane (4% in pure oxy-gen) and perfused with 4% paraformaldehyde in 100 mM phosphate buffer, pH 7.4. Brains were removed and stored in fixative overnight, kept in 20% sucrose in phosphate buffer for an additional 24 h and then frozen with cold ice. All steps of sectioning, imaging and data analysis were conducted blindly and performed by two different operators. Brains were cut with a freezing microtome into 30-μm-thick coronal sections that were serially collected. One out of 12 sections from the telencephalon were used for immunohistochemistry for HA (rabbit polyclonal anti-HA Ab, 1:500, Cell Signaling). Brain sections were incubated overnight at 4° C. with a primary antibody and for 2 h with an HRP-conjugated anti-rabbit secondary antibody (1:200, Jackson

Immunoresearch,). Detection was performed using the TSA Cyanine 3 Plus Evaluation Kit (Perkin Elmer).

### Protein Expression, Secretion and Western Blot Analysis

HEK293T cells were transfected with the following vectors: Igk-TATk-GFP, Igk-TATk-CDKL5, GFP and CDKL5 using Lipofectamine 3000 (Invitrogen) transfection reagent according to the manufacturer's instructions. Transfected cells were grown for 48 h in serum-free culture medium (Dulbecco's modified Eagle's medium supplemented with 2 mM of glutamine and antibiotics: penicillin, 100 U/ml; streptomycin, 100 mg/ml). The culture medium containing the secreted proteins was collected, centrifuged to pellet cell debris and filtered through 0.2 mm syringe filters. The culture medium was then transferred to Amicon Ultra Centrifugal Filters (Millipore) with 50 kDa molecular weight cut-off, and proteins underwent diafiltration according to the manufacturer's instructions. Western blot analysis was conducted on cell extracts in RIPA buffer (50 mM Tris-HCl, pH 7.4, 150 mM NaCl, 1% Triton-X100, 0.5% sodium deoxycholate, 0.1% SDS with 1 mM PMSF, and 1% protease and phosphatase inhibitor cocktail (Sigma-Aldrich)), and concentrated medium. Protein concentration was determined by the Lowry method (O. H. Lowry, N. J. Rosebrough, A. L. Farr, R. J. Randall, Protein measurement with the Folin phenol reagent. The Journal of biological chemistry 193, 265-275 (1951)). Proteins were subjected to electrophoresis on a BOLT bis-tris plus gel (Thermo Fisher Scientific) and transferred to a Hybond ECL nitrocellulose membrane (Amersham Life Science). The following primary antibody was used: rabbit polyclonal anti-HA (1:500, Cell Signaling). The following secondary antibody was used: anti-rabbit IgG (1:5000, Jackson ImmunoResearch) antibody. Densitometric analysis of digitized images was performed using Chemidoc XRS Imaging Systems and Image Lab™ Software (Bio-Rad).

### Behavioral Assays

All animal behavioural studies and analysis were performed blinded to treatment. Mice were allowed to habituate to the testing room for at least 1 h before the test, and testing was performed at the same time of day. A group of vehicle-treated Cdkl5 KO (−/Y; n=15) and wild-type (+/Y; n=15) mice were used as controls for behavioural tests.

Hind-limb clasping. Animals were suspended by their tail for 2 min and hind-limb clasping was assessed independently by two operators from video recordings. A clasping event is defined by the retraction of limbs into the body and toward the midline.

Marble burying. The marble burying test was performed by placing animals individually in a home-cage-like environment with 5 cm of unscented standard bedding material and 20 marbles (14.3 mm in diameter) arranged in a 4×5 matrix, and left undisturbed for 30 min. The number of marbles that were at least two-thirds buried at the end of the trial was counted.

Accelerating Rotarod assay. Before the first test session, animals were briefly trained at a constant speed of 5 rpm on the Rotarod apparatus (Ugo Basile) for 30 s. Thirty minutes later, testing was performed at an accelerating linear speed (5-35 rpm within 270 s+30 s max speed). Four testing trials with an inter-trial interval of 1 h were performed. The latency to fall from the rotating rod and the number of passive rotations (rotation in which the mouse does not perform any coordinated movement but is passively transported from the rotating apparatus) were recorded for each trial.

Open Field. In order to assess locomotion, animals were placed in the centre of a square arena (50×50 cm) and their behavior was monitored for 20 min using a video camera placed above the centre of the arena. Distinct features of locomotor activity, including total distance travelled, average locomotion velocity, and the time spent at the border, by the walls, and in the centre, were scored by EthoVision10XT software (Noldus Information Technology B.V., The Netherlands). The number of stereotypical jumps (repetitive beam breaks <1 s) in the corners of the arena were manually counted by a trained observer who was blind to the genotype and treatment. The test chambers were cleaned with 70% ethanol between test subjects.

Barnes Maze. Mice were trained to locate the target hole (with an underneath escape box) from 20 holes (5 cm diameter) evenly spaced around the perimeter of an elevated (60 cm above the floor) circular open field (100 cm diameter) (Ugo Basile,). The escape box was designated as an analogue to the hidden platform in the MWM, containing a ramp under the target hole so that mice could enter the escape box easily. Mice were initially placed in the centre of the arena covered by a dark cylinder, which was removed 10 s after the start of a trial. All mice were trained with three trials per day for three consecutive days with an inter-trial interval of 30 min. Mice that initially failed to locate the target hole within 3 min were gently guided to the target by the operator. The mouse was left in the escape box for 1 min and then returned to its home cage before the next training trial. On the next day mice were tested for the ‘probe trial’ (trial duration 90 s) in which the escape box was removed. All training trials and probe trials were videotaped for 3 min and the latency to enter the escape box/target hole and the number of errors were automatically scored by EthoVision10XT software (Noldus,).

Non-invasive assessment of sleep and breathing pattern. Hypnic and respiratory phenotypes of mice were assessed non-invasively with a validated technique based on whole-body plethysmography (WBP) (S. Bastianini et al., Accurate discrimination of the wake-sleep states of mice using non-invasive whole-body plethysmography. Scientific reports 7, 41698 (2017); V. Lo Martire et al., CDKL5 deficiency entails sleep apnoea's in mice. Journal of sleep research 26, 495-497 (2017). Briefly, each mouse was placed inside a WBP chamber flushed with air at 1.5 l/h for the first 8 h of the light period. The respiratory (WBP chamber pressure) signal was continuously recorded together with chamber humidity and temperature, digitized, and stored at 128 Hz, 4 Hz, and 4 Hz, respectively. The system was calibrated with a 100 μl micro-syringe (Hamilton, Reno, USA) at the end of each recording. The states of wakefulness, non-rapid-eye-movement sleep (NREMS) and rapid-eye-movement sleep (REMS) were scored based on inspection of the raw WBP signal with the investigators blinded to the animal's genotype. Quantitative analysis of breathing was restricted to stable sleep episodes 12 s because of the frequent occurrence of movement artefacts during wakefulness. Apnoea's were automatically detected as breaths with instantaneous total breath duration (TTOT) >3 times; the average TTOT for each mouse and sleep state, and detection accuracy were checked on raw recordings.

Statistical analysis. Data from single animals represented the unity of analysis. Results are presented as mean±standard error of the mean (±SE). Statistical analysis was performed using GraphPad Prism (version 6). All datasets were analyzed using the ROUT method (Q=1%) for the identification of significant outliers and the Shapiro-Wilk test for normality. Datasets with normal distribution were analyzed for significance using a one-way analysis of variance (ANOVA) with genotype as a factor. Post hoc multiple comparisons were carried out using the Fisher least significant difference (Fisher's LSD) test. Datasets with non-parametric distribution were analyzed using the Kruskal-Wallis test. Post hoc multiple comparisons were carried out using Dunn's multiple comparison test or the Mann-Whitney test. For the Rotarod assay and the learning phase of the Barnes maze, statistical analysis was performed using a repeated ANOVA. A probability level of p<0.05 was considered to be statistically significant.

### Results

### Successful Production of Secretable AAV-Igk-TATk-CDKL5 Vector for Gene Therapy

To experimentally verify the design efficiency of the newly-constructed AAV vector genomes, the secretion ability of the Igk-TATk-CDKL5 protein was evaluated. A transient plasmid DNA transfection assay using human embryonic kidney 293 (HEK293) cells was performed.

Western blot analysis detected TATK-CDKL5 protein (FIG. 2, line 3) in the culture medium 48 hours after transfection of HEK293 cells with the AAV-Igk-TATk-CDKL5 plasmid, indicating that Igk-TATκ-CDKL5 protein was efficiently secreted from transfected cells. As expected, CDKL5 without Igk-TATk was not present in the culture medium (FIG. 2, line 4).

High yield recombinant AAV vector production from the produced AAV vector plasmids using the standard adenovirus-free three plasmid transfection system (data not shown) was also confirmed.

### Effect Of Gene Therapy with AAVPHP.B Igk-TATk-CDKL5 oR AAVPHP.B CDKL5 Vector in CDKL5 KO Mice

To test whether the effects of the loss of Cdkl5 in adult mice can be reversed, Cdkl5 KO male mice aged 2 months were intravenously injected with AAVPHP.B_CDKL5 vector or AAVPHP.B_Igk-TATk-CDKL5 vector (FIG. 3). The effect of AAVPHP.B_CDKL5 vector or AAVPHP.B_Igk-TATk-CDKL5 vector at the dose of 5×10e11 vg was compared. The effects of treatment were evaluated 30 and 60 days post-injection (see Treatment schedule; FIG. 3). A group of vehicle-treated Cdkl5 KO (−/Y) and wild-type (+/Y) mice were used as controls for behavioural tests. The behavioural tests were repeated in two post-injection time windows to assess whether the effect of CDKL5 bioavailability in the brain increases with time. Some behavioural tests that could be performed on the same animal only once, were performed either at 30 days (1M) or 60 days (2M) after administration (see FIG. 3).

### Effect on Body Weight

In order to evaluate the presence of a toxic effect due to viral infection and/or CDKL5 expression, the body weight of treated Cdkl5 KO mice in comparison with vehicle-treated Cdkl5 KO mice was evaluated. Body weight for Cdkl5 KO mice was recorded prior to injection and 30 and 90 days after. No differences in body weight between treated and age-matched vehicle-treated mice at 1-3 months after treatment were observed (FIG. 4), suggesting that CDKL5 expression did not affect animal well-being.

### Effect on Autistic-Like Features

Loss of Cdkl5 function in Cdkl5 KO mice is associated with autistic-like (ASD-like) phenotypes, analyzed through home-cage social behaviours (marble burying; C. Fuchs et al., Heterozygous CDKL5 Knockout Female Mice Are a Valuable Animal Model for CDKL5 Disorder. Neural plasticity 2018, 9726950 (2018). The marble burying test was used to evaluate exploration and environmental interest. Cdkl5 KO (−/Y) mice buried a significantly lower number of marbles compared to wild-type (+/Y) mice (FIG. 5). Thirty days after treatment (1M) with AAVPHP.B_CDKL5 or AAVPHP.B_Igk-TATk-CDKL5 vectors Cdkl5 KO mice buried a higher number of marbles compared to vehicle-treated Cdkl5 KO mice (FIG. 5). 60 days after treatment the number of buried marbles became even higher (FIG. 5), suggesting a time-dependent effect of CDKL5 expression on brain function. Importantly, the greatest improvement was achieved through treatment with TATk-CDKL5, suggesting an increased CDKL5 bio distribution induced by TATk-CDKL5.

### Effect on Impaired Motor Function

We assessed motor function of treated Cdkl5 KO mice on an accelerating rotarod assay. Mice were tested on a rotating rod for 4 trials with an inter-trial interval of 1 h, and the frequency of passive rotations (number of passive rotations/sec), rotations in which the mouse does not perform any coordinated movement and is passively transported from the rotating apparatus, were evaluated. The test was performed 30 and 60 days after treatment. Cdkl5 KO mice showed significantly more passive rotations during the four trials in both tests (1M and 2M) in comparison with wild-type mice (FIG. 6). Notably, rotarod performance improved in Cdkl5 KO mice treated with AAVPHP.B_Igk-TATk-CDKL5 vector; in particular, in the second trial (2M) the number of passive rotations reduced and became similar to those of wild-type mice (FIG. 6). In contrast, only a small improvement in motor coordination was noted in Cdkl5 KO mice treated with AAVPHP.B_CDKL5 vector (FIG. 6). This evidence suggests that TATk-CDKL5 protein is more effective at improving impaired motor function in Cdkl5 KO mice.

### Effect on Stereotypic Behavior

In addition to motor dysfunction, stereotypic movements characterize Cdkl5 KO mice Fuchs and CDKL5 patients N. Bahi-Buisson, T. Bienvenu, CDKL5-Related Disorders: From Clinical Description to Molecular Genetics. Molecular syndromology 2, 137-152 (2012). Therefore, we evaluated stereotypic behavior in treated Cdkl5 KO mice by counting the number of repetitive jumps in the corners of the open-field arena. Cdkl5 KO mice showed an increased number of stereotypical jumps compared to wild-type mice (FIG. 7). The number of stereotypical jumps was drastically reduced in Cdk15 KO mice treated with AAVPHP.B-_CDKL5 or AAVPHP.B_Igk-TATk-CDKL5 vectors (FIG. 7). In particular, in Cdk15 KO mice treated with AAVPHP.B_Igk-TATk-CDKL5 vector the number of jumps became similar to that of wild-type mice, indicating a better efficacy of treatment with TATk-CDKL5 regarding stereotypic behaviour.

Finally, in order to examine the effect of gene therapy on motor stereotypies, mice were tested for hind-limb clasping before and after the 30 days of treatment (FIG. 8). While wild-type (+/Y) mice exhibited hind-limb clasping for a low proportion of the time, Cdkl5 KO (−/Y) mice spent about ⅓ of the test session in the clasping position (FIG. 8). Cdkl5 KO mice treated with AAVPHP.B_CDKL5 or AAVPHP.B_Igk-TATk-CDKL5 vectors showed a decrease in clasping (FIG. 8), indicating a treatment-induced improvement of motor stereotypies. Importantly, Cdkl5 KO mice treated with AAVPHP.B_Igk-TATk-CDKL5 vector showed a greater and significant decrease in clasping time, indicating that gene therapy with Igk-TATk-CDKL5 has a greater positive impact on the stereotypic behavior that is due to loss of Cdkl5 expression.

### Effect on Hyperactivity

Cdkl5 KO mice showed an increased hyperactivity in an open-field arena They travelled significantly longer distances and moved at a higher average speed compared to wild-type mice (FIG. 9), indicating elevated locomotor activity. We found that Cdkl5 KO mice treated with AAVPHP.B_CDKL5 or AAVPHP.B_Igk-TATk-CDKL5 vectors showed a tendency, albeit not statistically significant, to reduce these behaviours to a level comparable with that of wild-type mice (FIG. 9). The greatest improvement, also in this case, was obtained 60 days after treatment with TATk-CDKL5.

### Effect on Impaired Learning and Memory Performance

Learning and memory was evaluated 30 days after treatment using the Barnes maze. We found that, while vehicle and AAVPHP.B_CDKL5 treated Cdkl5 KO (−/Y) mice took longer to locate the target hole compared to control (+/Y) mice, AAVPHP.B_Igk-TATk-CDKL5 treated Cdkl5 KO (−/Y) mice underwent an improvement in their learning ability (FIG. 10). Cdkl5 KO mice treated with AAVPHP.B_Igk-TATk-CDKL5 vector, similarly to wild-type mice, exhibited a faster learning improvement during the first and second day of testing within the same day of trials (FIG. 10, lower panel). No improvement in learning was observed during the third day of testing, since mice remembered the position of the right hole from the first trial (data not shown).

Twenty-four hours after the last day of training, a probe test for reference memory was conducted. An impairment of short-term memory was observed in Cdkl5 KO mice, with an increase in the latency to find the target hole and an increase in the number of errors before finding the target hole (FIG. 11). Cdkl5 −/Y mice treated with AAVPHP.B_Igk-TATk-CDKL5 vector showed an improvement in latency to find the hole and a significant improvement in the number of errors before detecting the target hole (FIG. 11).

These results indicate an effect of TATk-CDKL5 therapy on learning and memory performance.

### Effect on Breathing Disturbance

Using whole-body plethysmography (WBP), we recently found that sleep apnoea's occur more frequently in Cdkl5 KO than in control mice (G. Gao et al., Clades of Adeno-associated viruses are widely disseminated in human tissues. J Virol 78, 6381-6388 (2004)) (FIG. 12), indicating a breathing disturbance during sleep in Cdkl5 KO mice. Treatment with AAVPHP.B_Igk-TATk-CDKL5 led to a drastic reduction in the number of apnoea's during REM sleep that became similar to that of control mice (FIG. 12).

### Evaluation of the Efficiency of AAV Vector Transduction

After sacrifice, brains and livers from treated Cdkl5 KO mice were collected. To assess the efficiency of vector transduction, we performed a SYBR Green-based quantitative PCR analysis on the samples to determine vector genome (vg) copy numbers per diploid genomic equivalent (i.e., vg/cell) in collected tissues, using vector genome-specific primers and the primers specific for the mouse agouti gene, which we used as an endogenous control for data normalization. Our results indicate that AAVPHP.B_Igk-TATk-CDKL5 and AAVPHP.B_Igk-TATk-CDKL5 vectors had similar brain and liver transduction efficiency (Table 1). Similar CDKL5 RNA levels in the brain and liver of AAVPHP.B_Igk-TATk-CDKL5 treated mice in comparison with those of AAVPHP.B_Igk-TATk-CDKL5 treated mice (FIG. 13) confirmed the transduction efficiency.

### Assessment of Biodistribution of Replaced Protein in the Brain

The efficiency of CDKL5 protein replacement in the brain was analyzed using immunohistochemistry. Although the AAVPHP.B_Igk-TATk-CDKL5 vector showed a similar infection efficiency in comparison with the AAVPHP.B_CDKL5 vector (see Table 1), we found that the AAVPHP.B_Igk-TATk-CDKL5 vector led to a higher CDKL5 protein replacement due to secretion and transduction into the neighbour cells of the TATk-CDKL5 protein (FIGS. 14 and 15).

### Development of Proof of Concept Therapies for Genetic Brain Disorders

Monogenic diseases provide the unique opportunity to use therapies, such as gene therapy, that do not require knowledge of the molecular and cellular effects of the mutated gene. Gene therapy consists in the replacement of a mutated gene with a healthy copy of the gene. However, the gene delivery problem, in particular to the CNS, has not yet been solved. We found that the AAV vectors carrying the IgK-TATk-CDKL5 transgene machinery showed a higher therapeutic effect compared to the CDKL5 vector devoid of the IgK-TATk, corroborating the utility and power of the IgK-TATk-CDKL5 approach.

We are confident that this study has provided a first proof of principle that an innovative gene therapy approach, based on the unique advantages of the IgK-TATk-CDKL5 transgene, can enhance the efficiency of a gene therapy for CDKL5 disorder. The compelling feature of our approach is that brain cells that fail to receive the therapeutic gene can also be transduced via a bystander effect of TATk-CDKL5, resulting in enhanced therapeutic effects.

Such exciting results imply that this approach may become a powerful tool for the cure of CDD, and could open avenues to gene therapy development for other monogenic diseases based on the unique and compelling properties of the Igk-TATk-fusion protein approach.

TABLE 1

| | CDKL5 | TATk-CDKL5 |
|---|---|---|
| Cortex | 0.14 ± 0.01 | 0.16 ± 0.08 |
| Cerebellum | 0.54 ± 0.09 | 0.56 ± 0.28 |
| Liver | 10.70 ± 1.26 | 16.83 ± 7.47 |

Brain and Liver transduction following intravenous injection of AAVPHP.B_CDKL5 (n=4) and AAVPHP.B_Igk-TATk-CDKL5 (n=4) vectors. Vector genome dissemination to the brain (cortex and cerebellum) and liver was assessed by qPCR. Values represent mean±SEM.

1. A gene therapy vector comprising a first nucleic acid coding for a secretory leader sequence, operatively linked to a second nucleic acid coding for a protein transduction domain (PTD), operatively linked to a third nucleic acid coding for a therapeutic protein.

2. The gene therapy vector of claim 1 wherein said vector is an adeno-associated viral vector (AAV).

3. The gene therapy vector of claim 2 wherein said vector is a vector has an optimal serotype for CNS gene therapy.

4. The gene therapy vector of claim 3 wherein said AAV vector is one of: AAV1, AAV2, AAV4, AAV5, AAV8, AAV9, AAV10, AAV11, AAV12, AAV13.

5. The gene therapy vector of the invention according to claim 4 wherein said vector comprises a nucleotide construct coding for a CBh promoter a first nucleotide sequence coding for a secretory leader sequence, operatively linked to a second nucleic acid coding for a protein transduction domain (PTD), operatively linked to a third nucleic acid coding for a therapeutic protein and a SV40 polyadenylation signal between two AAV2 inverted terminal repeats (ITRs).

6. The gene therapy vector of claim 1 wherein said secretory leader sequence is one of mouse IgK, Human OSM, VSV-G, Human IgG2 H, BM40, Secrecon, Human IgKVIII, CD33, Tpa, Human Chymotrypsinogen, Human trypsinogen-2, Human IL-2, Gaussia luc, Albumin(HSA), Influenza Haemagglutinin or Human insulin leader sequences.

7. The gene therapy vector of claim 6 wherein said secretory leader sequence has a sequence as defined by one of SEQ ID NO 1-16.

8. The gene therapy vector of claim 1 wherein said PTD is one of TATk, MPG, Pep-1, ARF(1-22), BPrPr(1-30), MAP, p28, VT5, C105Y, M918, DPV3, Human lactoferrin leader sequences.

9. The gene therapy vector of claim 8 wherein said PTD has a sequence as defined by one of SEQ ID NO 17-29.

10. The gene therapy vector of 1 wherein said vector is an AAV vector, said secretory leader sequence is mouse IgK and said PTD is TATk.

11. The gene therapy vector of claim 1 wherein said therapeutic protein is a protein for CNS therapy.

12. The gene therapy vector of claim 11 wherein said therapeutic protein is one of human: CDKL5, MECP2, FOXG1, SCN1A, NLGN3, SHANK3, ASPA, PAH.

13. (canceled)

14. A method of treating monogenic disease comprising administering the gene therapy vector of claim 1 to a subject in need thereof.

15. A method of treating a CNS disease comprising administering the gene therapy vector of claim 1 to a subject in need thereof.

16. The method of claim 15, wherein the CNS disease affects the brain.

17. The method according to claim 16 wherein said disease affecting the brain is one of FXS, Dravet syndrome, ASD, Phelan-McDermid syndrome or Rett Syndrome.

18. A viral particle consisting of a viral capsid and a gene therapy vector comprising a first nucleic acid coding for a secretory leader sequence, operatively linked to a second nucleic acid coding for a protein transduction domain (PTD), operatively linked to a third nucleic acid coding for a therapeutic protein.

19. The viral particle of claim 18 wherein said capsid is an AAV capsid and said gene therapy vector is an adeno-associated viral vector (AAV).

20. The viral particle of claim 19 wherein said vector is a vector has an optimal serotype for CNS gene therapy.

21. The viral particle of claim 20 wherein said wherein said AAV capsid is one of AAV-PHP, AAV9, AAV-BR1, AAV-Retro capsid.

22. The viral particle of claim 21 wherein said AAV-PHP capsid is AAV-PHP.B, AAV-PHP.eB, AAV-PHP.S or AAV-PHP.A.

23. The viral particle of claim 18 wherein said secretory leader sequence is one of mouse IgK, Human OSM, VSV-G, Human IgG2 H, BM40, Secrecon, Human IgKVIII, CD33, Tpa, Human Chymotrypsinogen, Human trypsinogen-2, Human IL-2, Gaussia luc, Albumin (HSA), Influenza Haemagglutinin or Human insulin leader sequences.

24. The viral particle of claim 23 wherein said secretory leader sequence has a sequence as defined by one of SEQ ID NO 1-16.

25. The viral particle of claim 18 wherein said PTD is one of TATk, MPG, Pep-1, ARF(1-22), BPrPr(1-30), MAP, p28, VT5, C105Y, M918, DPV3, Human lactoferrin leader sequences.

26. The viral particle of claim 25 wherein said PTD has a sequence as defined by one of SEQ ID NO 17-29.

27. The viral particle of claim 18 wherein said vector is an AAV vector, said secretory leader sequence is mouse IgK and said PTD is TATk.

28. The viral particle of claim 18 wherein said therapeutic protein is a protein for CNS therapy.

29. The viral particle of claim 28 wherein said therapeutic protein is one of human: CDKL5, MECP2, FOXG1, SCN1A, NLGN3, SHANK3, ASPA, PAH.

30. (canceled)

31. A method of treating monogenic disease comprising administering the viral particle of claim 18 to a subject in need thereof.

32. A method of treating CNS disease comprising administering the viral particle of claim 18 to a subject in need thereof.

33. The method according to claim 32 wherein said gene therapy treatment is of a disease affecting the brain.

34. The method according to claim 33 wherein said disease affecting the brain is one of FXS, Dravet syndrome, ASD, Phelan-McDermid syndrome or Rett Syndrome.

35. A pharmaceutical composition comprising the viral particle according to claim and a pharmaceutically acceptable carrier.

36. (canceled)

37. A method of treating monogenic disease comprising administering the composition of claim 35 to a subject in need thereof.

38. A method of treating a CNS disease comprising administering the composition of claim 35 to a subject in need thereof.

39. The method of claim 38, wherein the disease affects the brain.

40. The method according to claim 39 wherein said disease affecting the brain is one of CDKL5 deficiency disorder, Dravet syndrome, Phenylketonuria, ASD, Phelan-McDermid syndrome, Canavan disease or Rett Syndrome.

41. The method according to claim 35 for administration by systemic injection, central nervous system delivery or aerosol/nasal delivery.

42. The method according to claim 41 for intravenous injection administration, intraparenchymal administration in particular areas of the brain such as intracerebroventricular, cisternal, lumbar or intrathecal administration, or intra-arterial injection administration, or for direct administration into the cerebrospinal fluid.
